(19) United States
(12) Patent Application Publication
(10) Pub. No.: US 2023/0307085 A1
(43) Pub. Date: Sep. 28, 2023

(54) METHOD AND SYSTEM FOR PREDICTING MUTATIONS IN RIBONUCLEIC ACID STRAINS

(71) Applicant: Wipro Limited, Bangalore (IN)

(72) Inventors: Manoj MADHUSUDHANAN, Bangalore (IN); Sreekumar CHOYARMADATHIL, Bangalore (IN); Mayank PRAKASH, Bhagalpur (IN); Rohan MADHUSUDHANAN, Bhagalpur (IN)

(21) Appl. No.: 17/804,895

(22) Filed: Jun. 1, 2022

(30) Foreign Application Priority Data
Mar. 22, 2022 (IN) 202241015818

(51) Int. Cl. G16B 5/00; G16B 35/00; G16B 30/10; G16B 40/20

(52) U.S. Cl. CPC G16B 5/00; G16B 35/00; G16B 30/10; G16B 40/20

(57) ABSTRACT

Disclosed herein is method and a system for predicting mutations in Ribonucleic acid (RNA) strains. In an embodiment, a similarity between a new viral RNA strain and reference RNA strains is determined. Further, a strain score for the new viral RNA strain is calculated based on the similarity between the new viral RNA strain and the reference RNA strains. Subsequently, mutation sites for the new viral RNA strain are identified by generating spatial nearness data corresponding to the reference RNA strains based on comparison between the strain score of the new viral RNA strain and the reference RNA strains. Finally, mutations of the new viral RNA strain are predicted by performing a generative modelling of a sequence of the new viral RNA strain with reference to the mutation sites of the new viral RNA strain.

### TECHNICAL FIELD

The present disclosure, in general, relates to study of viral pathogens, and particularly to a method and system for predicting mutations in Ribonucleic Acid (RNA) strains using Artificial Intelligence (AI) models.

### BACKGROUND

Identification of emerging infections and harmful viral strains before zoonotic transmission to human hosts is a key challenge. After zoonotic transmission, delay in identification of a viral epidemic leads to widespread economic and social distress. The strain can spread unidentified across country borders and potentially become a pandemic. This also leads to delays in releasing vaccines against the strain, leading to a prolonged epidemic period.

Identification of patterns that make a strain more infectious and/or fatal during cell entry and replication phases is key to efforts for vaccine development. In-vitro solutions for viral strains identification and prediction for vaccine development are extremely slow and cost intensive due to the requirement of physical cultures to be grown. Current in-silico experiments are extremely parallel and fast relative to in-vitro, however they require subject experts to define the parameters for the simulations. These solutions cannot simulate the large number of possible variations in the viral genome in reasonable time to study the effects of mutations.

The information disclosed in this background of the disclosure section is only for enhancement of understanding of the general background of the invention and should not be taken as an acknowledgement or any form of suggestion that this information forms the prior art already known to a person skilled in the art.

### SUMMARY

Disclosed herein is a method for predicting mutations in Ribonucleic acid (RNA) strains. The method comprises determining, by a prediction system, a similarity between a new viral RNA strain and one or more reference RNA strains. Further, the method comprises calculating a strain score for the new viral RNA strain based on the similarity between the new viral RNA strain and the one or more reference RNA strains. Thereafter, the method comprises identifying one or more mutation sites for the new viral RNA strain by generating spatial nearness data corresponding to the one or more reference RNA strains based on comparison between the strain score of the new viral RNA strain and the one or more reference RNA strains. Finally, the method comprises predicting one or more mutations of the new viral RNA strain by performing a generative modelling of a sequence of the new viral RNA strain with reference to the one or more mutation sites of the new viral RNA strain.

Further, the present disclosure relates to a prediction system for predicting mutations in Ribonucleic acid (RNA) strains. The prediction system comprises a processor and a memory. The memory is communicatively coupled to the processor and stores processor-executable instructions, which on execution, cause the processor to determine a similarity between a new viral RNA strain and one or more reference RNA strains. Further, the instructions cause the processor to calculate a strain score for the new viral RNA strain based on the similarity between the new viral RNA strain and the one or more reference RNA strains. Thereafter, the instructions cause the processor to identify one or more mutation sites for the new viral RNA strain by generating spatial nearness data corresponding to the one or more reference RNA strains based on comparison between the strain score of the new viral RNA strain and the one or more reference RNA strains. Finally, the instructions cause the processor to predict one or more mutations of the new viral RNA strain by performing a generative modelling of a sequence of the new viral RNA strain with reference to the one or more mutation sites of the new viral RNA strain.

Furthermore, the present disclosure relates to a non-transitory computer readable medium including instructions stored thereon that when processed by at least one processor, cause a prediction system to perform operations comprising determining a similarity between a new viral RNA strain and one or more reference RNA strains. Further, the instructions cause the processor to calculate a strain score for the new viral RNA strain based on the similarity between the new viral RNA strain and the one or more reference RNA strains. Thereafter, the instructions cause the processor to identify one or more mutation sites for the new viral RNA strain by generating spatial nearness data corresponding to the one or more reference RNA strains based on comparison between the strain score of the new viral RNA strain and the one or more reference RNA strains. Finally, the instructions cause the processor to predict one or more mutations of the new viral RNA strain by performing a generative modelling of a sequence of the new viral RNA strain with reference to the one or more mutation sites of the new viral RNA strain.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 shows an overview of functioning of a prediction system in accordance with some embodiments of the present disclosure.

FIG. 2 shows a detailed block diagram of a prediction system in accordance with some embodiments of the present disclosure.

FIG. 3 shows a flowchart illustrating a method for predicting mutations in Ribonucleic acid (RNA) strains, in accordance with some embodiments of the present disclosure.

FIG. 4 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the scope of the disclosure.

The terms “comprises”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

In an embodiment, the present disclosure proposes a method that helps to identify viral pathogens even before the infection begins in human hosts. This pre-emptive method allows vaccine development to begin before the disease becomes an epidemic. An epidemic is defined when a rate of infection ‘R’ is more than 1. In an embodiment, the present invention uses machine learning algorithms to improve the speed of search by parallelizing the operation of pattern matching to identify mutations of importance, without always relying on heuristics. The used machine learning algorithms are effective in optimizing the parameters and converging to an optimal solution and generalize well for new data. The present invention creates an early warning system against potential pathogens and provides ample time for pharmaceutical companies and public health organizations to prepare for vaccine development and mitigate public health risk. By identifying the target viral strain, vaccine research costs and time to market reduces significantly. With this approach, mutations that occur in an RNA can be predicted and the corresponding vaccine candidate against the mutations can be developed and evaluated.

In an embodiment, the proposed method aims for early identification of viral strains that have potential to turn into an epidemic. Also, the proposed method improves the response time in vaccine development and consequently shortens time to market, making the vaccines available for clinical trials earlier.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

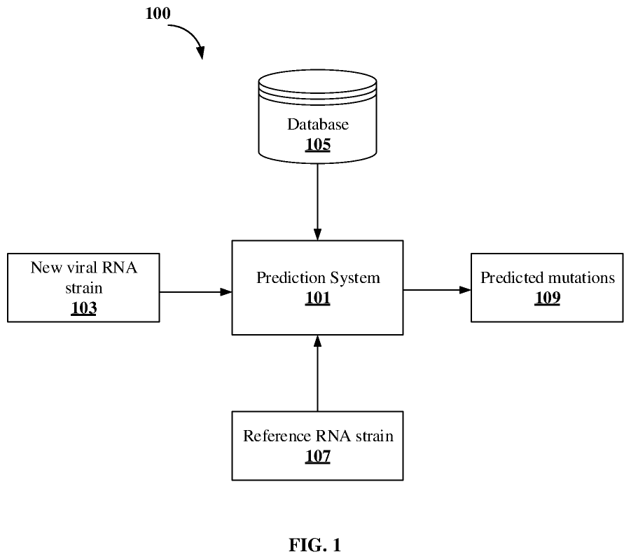

FIG. 1 shows an overview of functioning of a prediction system, in accordance with some embodiments of the present disclosure.

In an embodiment, a prediction system 101, which may be configured and used for predicting the mutations in the new viral Ribonucleic acid (RNA) strains 107, may be provided with one or more reference RNA strains 107 and a database 105. The one or more reference RNA strains 107 are the RNA strains belonging to a same family of the new viral RNA strain 103. The database 105 may comprise data related to the one or more reference RNA strains 107 from earlier pandemic/epidemic. As an example, the database 105 may be the databases provided by National Center for Biotechnology Information (NCBI), Global Initiative on Sharing All Influenza Data (GISAID) etc. The prediction system 101 may be any computing system such as, without limiting to, a desktop computer, a laptop, a smartphone and the like.

In an embodiment, upon receiving the new viral RNA strain 103, the prediction system 101 calculates a strain score for the new viral RNA strain 103 based on the similarity between the new viral RNA strain 103 and the one or more reference RNA strains 107. The strain score for the one or more reference RNA strains 107 is determined by normalizing a normalized infectivity score and a normalized mortality score using a Euclidean norm. The infectivity and mortality score are calculated using an infectivity metrics and a mortality metrics of the one or more reference RNA strains 107 based on their analysis from earlier epidemics. The infectivity metrics corresponds to a Basic Reproduction Number (R₀) data of the one or more reference RNA strains 107 from the earlier epidemics. The mortality metrics corresponds to Case Fatality Ratio (CFR) data of the one or more reference RNA strains 107 from earlier epidemics.

In an embodiment, subsequent to calculating the similarity score, the prediction system 101 identifies one or more mutation sites for the new viral RNA strain 103 by generating a spatial nearness data corresponding to the one or more reference RNA strains 107 based on comparison between the strain score of the new viral RNA strain 103 and the one or more reference RNA strains 107. The spatial nearness data is a measure of structural similarity of reference RNA sequences of the one or more reference RNA strains 107 with one another.

In an embodiment, the prediction system 101 predicts the one or more mutations of the new viral RNA strain 103 by performing a generative modelling of a sequence of the new viral RNA strain 103 with reference to the one or more mutation sites of the new viral RNA strain 103. The generative model takes the viral RNA sequence of the new viral RNA strain 103, information of possible mutation sites and the experimental data to generate a next generation of the new viral RNA strain 103 with mutations. Further, the prediction system calculates a vibrational entropy and an overall structural stability of the mutated RNA sequence to identify one or more stable mutations.

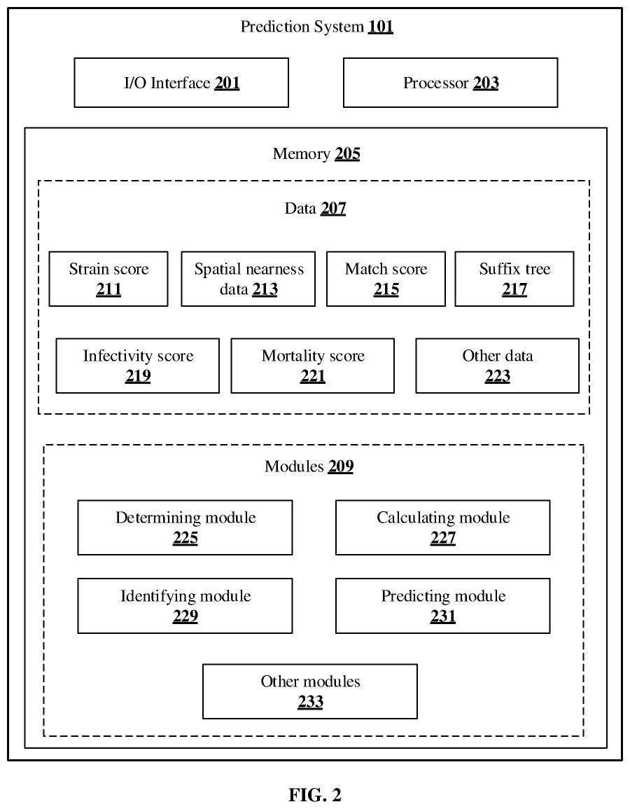

FIG. 2 shows a detailed block diagram of a prediction system 101 in accordance with some embodiments of the present disclosure.

In an embodiment, the prediction system 101 may include an I/O interface 201, a processor 203 and a memory 205. The processor 203 may be configured to perform one or more functions of the prediction system 101 for predicting mutations in Ribonucleic acid (RNA) strains, using the data 207 and the one or more modules 209 in stored in a memory 205 of the prediction system 101. In an embodiment, the memory 205 may store data 207 and one or more modules 209.

In an embodiment, the data 207 may be stored in the memory 205 may include, without limitation, strain score 211, a spatial nearness data 213, a match score 215, a suffix tree 217, an infectivity score 219, a mortality score 221 and other data 223. In some implementations, the data 207 may be stored within the memory 205 in the form of various data structures. Additionally, the data 207 may be organized using data models, such as relational or hierarchical data models. The other data 223 may include various temporary data and files generated by the one or more modules 209.

In an embodiment, the strain score 211 is a score assigned for the one or more reference RNA strains 107 by normalizing a normalized infectivity score and a normalized mortality score using an Euclidean norm. The strains from the set of reference RNA strains 107 that closely resemble the new viral RNA strain 103 are identified based on the match scores 215 and strain scores 211 associated with each of the one or more RNA strains.

In an embodiment, the spatial nearness data 213 is a measure of structural similarity of reference RNA sequences of the one or more reference RNA strains 107 with one another. The reference RNA sequences of the one or more reference RNA strains 107 are ordered spatially based on the spatial nearness data 213.

In an embodiment, the match score 215 is a calculated between the viral RNA sequence of the new viral RNA strain 103 and the reference RNA sequence of the reference RNA strain 107 by aggregating the match proportion of all the structural proteins. The strains from the set of reference RNA strains 107 that closely resemble the new viral RNA strain 103 are assigned with a higher match score.

In an embodiment, the suffix tree 217, which is a tree-based data structure, is a compressed tree containing all the suffixes of the given text as their keys and positions in the text as their values. The suffix tree 217 is constructed for every structural protein in the viral RNA sequence of the new viral RNA strain 103 and the reference RNA sequences of the one or more reference RNA strains 107.

In an embodiment, the infectivity score 219 is the score calculated by weighing the match score between the viral RNA sequence of the new viral RNA strain 103 and the reference RNA sequence of the one or more reference RNA strains 107 with the infectivity metrics data.

In an embodiment, the mortality score 221 is the score calculated by weighing the match score between the viral RNA sequence of the new viral RNA strain 103 and the reference RNA sequence of the one or more reference RNA strain 107 with the mortality metrics data.

In an embodiment, the data 207 may be processed by the one or more modules 209 of the prediction system 101. In some implementations, the one or more modules 209 may be communicatively coupled to the processor 203 for performing one or more functions of the prediction system 101. In an implementation, the one or more modules 209 may include, without limiting to, a determining module 225, a calculating module 227, an identifying module 229, a predicting module 231 and other modules 233.

As used herein, the term module may refer to an Application Specific Integrated Circuit (ASIC), an electronic circuit, a hardware processor (shared, dedicated, or group) and memory that execute one or more software or firmware programs, a combinational logic circuit, and/or other suitable components that provide the described functionality. In an implementation, each of the one or more modules 209 may be configured as stand-alone hardware computing units. In an embodiment, the other modules 233 may be used to perform various miscellaneous functionalities of the prediction system 101. It will be appreciated that such one or more modules 209 may be represented as a single module or a combination of different modules.

In an embodiment, the determining module 225 may be configured for determining a similarity between a new viral RNA strain 103 and one or more reference RNA strains 107. In an embodiment, the calculating module 227 may be configured for calculating a strain score 211 for the new viral RNA strain 103 based on the similarity between the new viral RNA strain 103 and the one or more reference RNA strains 107. In an embodiment, the identifying module 223 may be configured for identify one or more mutation sites for the new viral RNA strain 103 by generating spatial nearness data 213 corresponding to the one or more reference RNA strains 107 based on comparison between the strain score 211 of the new viral RNA strain 103 and the one or more reference RNA strains 107. In an embodiment, the predicting module 227 may be configured for predicting one or more mutations 109 of the new viral RNA strain 103 by performing a generative modelling of a sequence of the new viral RNA strain 103 with reference to the one or more mutation sites of the new viral RNA strain 103. The functioning of each module in the set of modules 209 is explained in detail in the below sections.

In an embodiment, the determining module 225 determines the similarity of the new viral RNA strain 103 with the one or more reference RNA strains 107 in the virus family. The determining module 225 determines the closest reference RNA strains 107 to the new viral RNA strain 103 and determines the top structural proteins in the new viral RNA strain 103 that contribute to its viral properties. In an embodiment, the determining module 225 identifies and collects viral RNA sequence of the new viral strain 103 and reference RNA sequences of the one or more reference RNA strains 107 from earlier pandemics/epidemics belonging to the same virus family. The viral RNA sequence of the new viral RNA 103 strain and the reference RNA strain sequences of the one or more reference RNA strains 107 are obtained from nucleotide sequence databases. The nucleotide sequence databases are provided by National Center for Biotechnology Information (NCBI), Global Initiative on Sharing All Influenza Data (GISAID) etc. Further, a timeline such as a year, a month etc., of the occurrence of the reference RNA strains 107 is also obtained from the nucleotide sequence databases.

In an embodiment, the calculating module 225 identifies and collects infectivity and mortality metrics of the one or more reference RNA strains 107 from earlier epidemics. The infectivity metric corresponds to Basic Reproduction Number (R₀) data of strains from earlier epidemics. R₀ refers to the number of individuals directly infected by an infectious person in a population where all individuals are susceptible to infection. If R₀ is greater than 1 it refers to an existing infection that causes more than one infection. The mortality metrics corresponds to Case Fatality Ratio (CFR) data of strains from earlier epidemics. The CFR refers to the proportion of the number of deaths from an infection or disease divided by the total number of individuals diagnosed with that infection or disease. In an embodiment, the calculating module 225 obtains the infectivity metric data and the mortality metric data for the one or more reference RNA strains 107 from various studies done previously in the field of emerging infectious diseases.

In an embodiment, a plurality of suffix trees 217 is created for viral RNA sequence of the new viral RNA strain 103 and reference RNA sequences of the one or more reference RNA strains 107. A suffix tree 217 may be constructed for every structural protein in the viral RNA sequence of the new viral RNA strain 103 and reference RNA sequences of the one or more reference RNA strains 107. In an embodiment, the structural proteins of the viral RNA sequence of the new viral RNA strain 103 are checked to determine if they are present in the reference RNA sequence of the reference RNA strain 107 so as to ensure that they belong to the same family.

The similarity of each structural protein of the viral RNA sequence of the new viral RNA strain 103 is checked with the corresponding structural protein of the reference RNA sequences of the reference RNA strain 107 using suffix trees 217. For every structural protein in the viral RNA sequence, the portion of the viral RNA sequence that matches with reference RNA sequence is called as an overlapping subsequence. The remaining portion of the viral RNA sequence is called as a non-overlapping subsequence, which is again matched with the remaining portion of the reference RNA sequence. This process is repeated until the complete viral RNA sequence for that structural protein is traversed. All the overlapping subsequences of the viral RNA that are at least nine nucleobases (or 3 amino acids) long are counted as matched against the corresponding structural protein of the reference RNA sequence.

The similarity between each structural protein of the viral RNA sequence of the new viral RNA strain 103 and the corresponding structural protein of the reference RNA sequence of the reference RNA strain 107 is determined. For every structural protein, the similarity i.e., the match proportion, is obtained as a ratio of matched base pairs (sum of overlapping subsequences length) in the viral RNA sequence of the new viral RNA strain 103 to total base pairs in the reference RNA sequence of the reference RNA strain 107 for that structural protein. In an embodiment, the match score 215 between the viral RNA sequence of the new viral RNA strain 103 and reference RNA sequence of the reference RNA strain 107 is calculated by aggregating the match proportion of all the structural proteins. This above mentioned steps are done using the algorithm 1 shown below.

Algorithm 1: Suffix tree comparison

```
ref_tree ← Reference protein suffix tree
ref_seq ← Reference protein sequence
tgt_seq ← Target protein sequence
i = 0
matched_substring ← empty string
substrings ← empty set
while i < length(tgt_seq) do
  Follow longest path in suffix tree for sequence tgt_seq[i...
  length(tgt_seq)]
  if longest_path == null then
    matched_substring = null
  else if length(longest_path) > 0 and longest_path terminates
  before leaf node
then
    i=i+length(longest_path)
    matched_substring = longest_path
  else:
    i=i+1
    matched_substring = longest_path
  add matched_substring to set substrings
min_len_strs = substrings where length(substring) > min_len for
all substrings
match_proportion = length(min_len_strs)/length(ref_seq)
```

In an embodiment, the calculating module 225 identifies and collects infectivity and mortality metrics of the one or more reference RNA strains 107 from the earlier epidemics. The infectivity metric corresponds to Basic Reproduction Number (R₀) data of strains from earlier epidemics. R₀ refers to the number of individuals directly infected by an infectious person in a population where all individuals are susceptible to infection. If R₀ is greater than 1, it refers to an existing infection that causes more than one infection. The mortality metrics corresponds to Case Fatality Ratio (CFR) data of strains from earlier epidemics. The CFR refers to the proportion of the number of deaths from an infection or disease divided by the total number of individuals diagnosed with that infection or disease. In an embodiment, the calculating module 225 obtains infectivity and mortality metric data for the one or more reference RNA strains 107 from various studies done previously in the field of emerging infectious diseases.

In an embodiment, the calculating module 225 calculates the infectivity score by weighing the match score between the viral RNA sequence of the new viral RNA strain 103 and the reference RNA sequence of the reference RNA strain 107 with the infectivity metrics data. The mortality score 221 is calculated by weighing the match score 215 between the viral RNA sequence of the new viral RNA strain 103 and the reference RNA sequence of the reference RNA strain 107 with the mortality metrics data. The normalized infectivity score is calculated by dividing the infectivity score 219 with the sum of infectivity metric data of the one or more reference RNA strains 107. Similarly, the normalized mortality score is calculated by dividing the mortality score 221 with the sum of mortality metric data of the one or more reference RNA strains 107. The strain score 211 of the one or more reference RNA strain 107 is determined by normalizing the normalized infectivity score and the normalized mortality score using a predefined Euclidean norm.

In an embodiment, the calculating module 225 determines the closest reference RNA strains 107 to the new viral RNA strain 103. The strains from the set of reference RNA strains 107 that closely resemble the new viral strain 103 are identified based on the match scores 215 and strain scores 211. The one or more reference RNA strains 107 having high match scores 215 with the new viral RNA strain 103 are considered similar to the new viral RNA strain 103. The one or more reference RNA strains 107 that have closer match scores 215 and strain scores 211 are considered similar to each other. This helps in determining the characteristics and the type of the new viral RNA strain 103. In an embodiment, the calculating module 225 determines the top contributing structural proteins in the new viral strain 103 and identifies the top two contributing structural proteins that are responsible for the current characteristics of the new viral RNA strain 103 based on the match proportion, match scores 215 and strain scores 211. This above mentioned steps are done using the algorithm 2 shown below.

Algorithm 2: Scoring

```
protein_scores ← empty set
infectivity_prior ← key value pair. One scalar for each epidemic
mortality_prior ← key value pair. One scalar for each epidemic
infectivity_score = 0
mortality_score = 0
For each reference epidemic strain:
  For reference protein suffix tree:
    match_score = tree_comparison( )
    infectivity_score = match_score * infectivity_prior[epidemic]
    mortality_score = match_score * mortality_prior[epidemic]
  infectivity_norm_score = infectivity_score/sum(infectivity_prior)
  mortality_norm_score = mortality_score/sum(mortality_prior)
  strain_score = Euclidean_norm({infectivity_norm_score, mortality_
  norm_score})
```

In an embodiment, the identifying module 227 orders the reference RNA sequences of the one or more reference RNA strains 107 temporally from oldest to newest reference RNA strain. The timeline such as year, month etc. of the occurrence of reference RNA strains 107 is obtained from the nucleotide sequence databases. The identifying module 227 generates spatial nearness data 213 for the reference RNA sequences based on comparison of the strain scores 211 of the one or more reference RNA strains 107 obtained previously. The spatial nearness data 213 is a measure of structural similarity of the reference RNA sequences of the one or more reference RNA strains 107 with one another. The reference RNA sequences of the one or more reference RNA strains 107 are ordered spatially based on the spatial nearness data 213. Ancestor for all the reference RNA sequences of the one or more reference RNA strains 107 is the reference RNA sequence of the reference strain, if information is available from nucleotide sequence databases, or the reference RNA sequence of the oldest reference RNA strain 107 by time. This is a fixed point and the spatial nearness of all the reference RNA sequences of the other reference RNA strains 107 are determined relative to the ancestor. The identifying module 227 generates a graph of temporal and spatial similarity between the reference RNA sequences of the one or more reference RNA strains 107 by iterating them over time and measuring the distance to the ancestor.

In an embodiment, the identifying module 227 groups the reference RNA sequences of the one or more reference RNA strains 107 by consecutive non-overlapping indices based on the graph of temporal and spatial similarity. Further, the identifying module 227 processes the reference RNA sequences of the one or more reference RNA strains 107 using an attention-based transformer model. The training samples of the attention-based transformer model include all combinations of reference RNA sequences of two nearest strains sampled from reference strain to the most recent reference RNA strain 107 in the graph. The attention-based transformer model predicts the mutation sites that can transform reference RNA sequence of an input strain into reference RNA sequence of an output strain. The attention-based transformer model is trained until the prediction loss no longer stops reducing. On convergence, the attention-based transformer model weights learn to identify conserved subsequences between the reference RNA sequences of two consecutive one or more reference RNA strains 107 temporally i.e., for example a reference RNA strain 107 that occurred at time t−1 and a reference RNA strain 107 that occurred at time t. The conserved subsequences are those regions in reference RNA sequence that have not undergone any mutation across several generations. The attention-based transformer model identifies the positions at which difference in sequence is observed from the original reference RNA sequences. These positions are possible mutation sites for the new viral strain 103. The above mentioned steps are done using the algorithm 3 and 4 shown below.

Algorithm 3: Strain lineage

strain_lineage:
time_sorted_strains ← set of strains ordered from oldest to newest in time
strain_lineage_list ← ascending_sort (time_sorted_strains) by
euclidean norm (strain scores)

Algorithm 4: Transformer data preparation

```
  i ← 0
  j ← 0
  for i, j from 0... length(strain_lineage_list)-1 such that j = i+1 do
    input_strain = strain_lineage_list[i]
    output_strain = strain_lineage_list[j]
  embedded_io ← empty set of tuples
  input_embedding ← null
  output_embedding ← null
  for strain in (input_strain, output_strain) do
    for i from 0...length(strain) do
      if i%2 == 0 then
        embedding[i] = sin(i/10000^{2D/Dmodel})
      else
        embedding[i] = cos(i/10000^{2D/Dmodel})
    if strain is input_strain do
      input_embedding = embedding
    else
output_embedding = embedding
where D is the dimension and Dmodel is the total embedding
dimensions
```

In an embodiment, the prediction module 229 predicts the possible RNA mutations of the new viral RNA strain using generative modelling. The prediction module 229 receives viral RNA sequence of the new viral strain 103 and experimental data on human RNA motifs that interact with proteins. The experimental data can be obtained from various databases of human RNA Binding Proteins (RBPs). The prediction module 229 also receives possible mutation sites information from the identification module 227. The prediction module 229 utilizes a generative model that takes the viral RNA sequence of the new viral RNA strain (Nth generation strain), the possible mutation sites information and experimental data to generate the next generation (N+1^{th} generation) of the new viral RNA strain 103 with mutations. The prediction module 229 creates a list of all possible amino acids from experimental data that could result in mutation at a given site of the viral RNA sequence defined by receptor binding domains. The prediction module 229 generates mutated RNA sequences of the viral RNA sequence of the new viral RNA strain 103 by introducing different possible amino acid structural changes at the possible mutation sites in the viral RNA sequence.

The prediction module 229 checks the overall structural stability of the mutated RNA sequences to find stable mutations. The prediction module 229 calculates the vibrational entropy and free energy for the mutated RNA sequences. Also, the prediction module 229 determines the RNA mutations with lowest free energy as those possibly can come into existence. The mutation sites (motifs) for the mutated RNA sequence identify zones of importance i.e., positions that can bring changes in function.

The invention can also be utilized to simulate antigenic shifts by setting the appropriate seed to visualize the effect of antigenic shift, which are a major source of zoonotic transmission

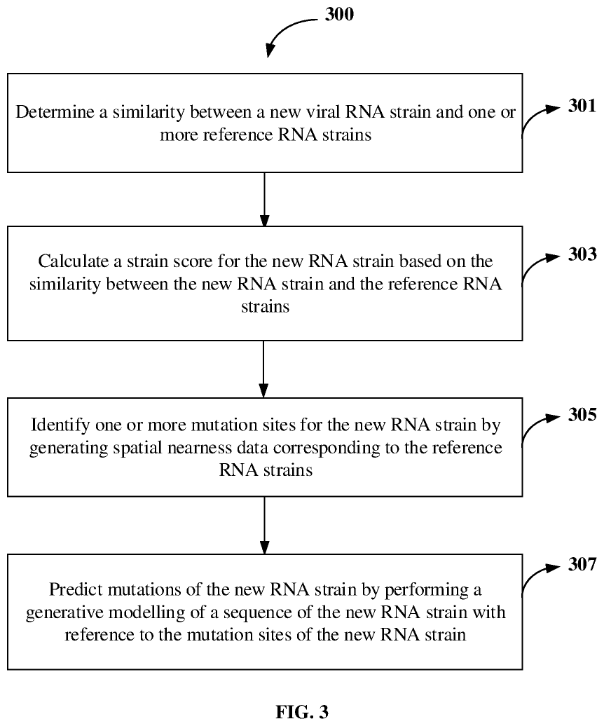

FIG. 3 shows a flowchart illustrating a method for predicting mutations in Ribonucleic acid (RNA) strains, in accordance with some embodiments of the present disclosure.

As illustrated in FIG. 3, the method 300 may include one or more blocks illustrating a method for predicting mutations in Ribonucleic acid (RNA) strains using a prediction system 101 illustrated in FIG. 2. The method 300 may be described in the general context of computer executable instructions. Generally, computer executable instructions can include routines, programs, objects, components, data structures, procedures, modules, and functions, which perform specific functions or implement specific abstract data types.

The order in which the method 300 is described is not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

At block 301, the method 300 includes determining, by the prediction system 101, a similarity between a new viral RNA strain 103 and one or more reference RNA strains 107. In an embodiment, the sequence of the new viral RNA strain 103 and a sequence of the one or more reference RNA strains 107 is collected. Further, a plurality of suffix trees is constructed, the suffix trees correspond to the sequence of the new viral RNA strain 103 and sequence of each of the one or more reference RNA strains 107. Thereafter, a match score for the new viral RNA strain 103 is determined by comparing the plurality of suffix trees to determine the similarity between the new viral RNA strain 103 and the one or more reference RNA strains 107.

At block 303, the method 300 includes calculating, by the prediction system 101, a strain score for the new viral RNA strain 103 based on the similarity between the new viral RNA strain 103 and the one or more reference RNA strains 107. In an embodiment, an infectivity metrics and a mortality metrics of the one or more reference RNA strains 107 from earlier epidemics is collected. In an embodiment, the strain score for the one or more reference RNA strains 107 is the score determined by normalizing the normalized infectivity score and the normalized mortality score using a Euclidean norm. In an embodiment, the infectivity metrics corresponds to a Basic Reproduction Number (R₀) data of the one or more reference RNA strains 107 and the mortality metrics corresponds to Case Fatality Ratio (CFR) data of strains from earlier epidemics.

At block 305, the method 300 includes identifying, by the prediction system 101, one or more mutation sites for the new viral RNA strain 103 by generating spatial nearness data corresponding to the one or more reference RNA strains 107 based on comparison between the strain score of the new viral RNA strain 103 and the one or more reference RNA strains 107. In an embodiment, sequences of each of the one or more reference RNA strains 107 is ordered based on the spatial nearness data. The spatial nearness data comprises a temporal similarity and a spatial similarity. In an embodiment, a pattern of the temporal similarity and the spatial similarity is determined using an Artificial Intelligence (AI) based attention transformer model and one or more mutation sites are identified by identifying differences in the pattern.

At block 307, the method 300 includes predicting, by the prediction system 101, one or more mutations of the new viral RNA strain 103 by performing a generative modelling of a sequence of the new viral RNA strain 103 with reference to the one or more mutation sites of the new viral RNA strain 103. In an embodiment, a plurality of human RNA Binding Protein (RBP) data is collected. The human RBP data provides an experimental data on human RNA motifs that interact with one or more proteins. In an embodiment, a mutated RNA sequence of the new viral RNA strain 103 is predicted using a generative model, the generative model takes the viral RNA sequence of the new viral RNA strain 103, information of possible mutation sites and the experimental data to generate a next generation of the new viral RNA strain 103 with mutations. In an embodiment, a vibrational entropy is calculated, and an overall structural stability of the mutated RNA sequence is checked to identify one or more stable mutations.

Computer System

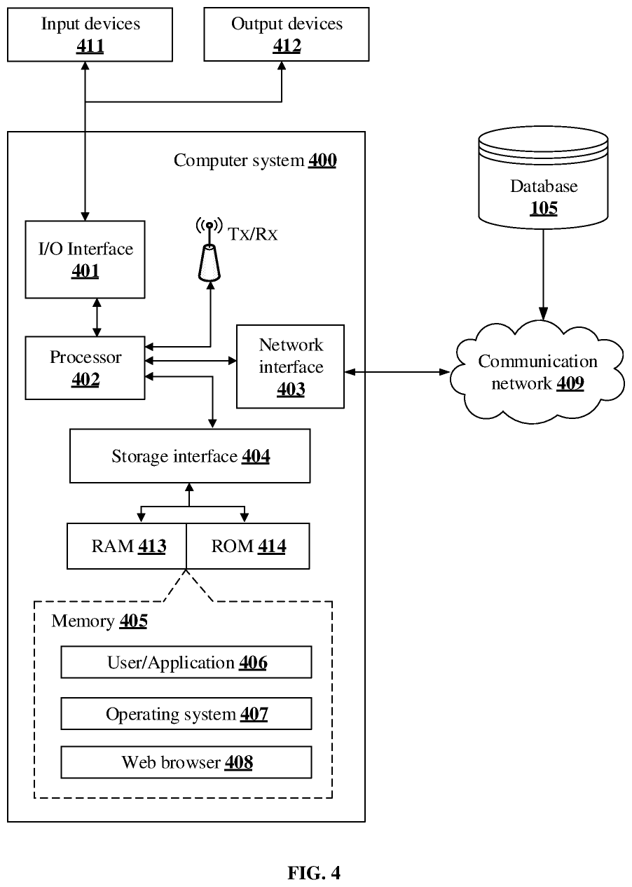

FIG. 4 illustrates a block diagram of an exemplary computer system 400 for implementing embodiments consistent with the present disclosure. In an embodiment, the computer system 400 may be the prediction system 101 illustrated in FIG. 2, which may be used for predicting mutations in Ribonucleic acid (RNA) strains. The computer system 400 may include a central processing unit (“CPU” or “processor” or “memory controller”) 402. The processor 402 may comprise at least one data processor for executing program components for executing user- or system-generated business processes. A user may include a researcher, a pathologist, a qualified medical professional or any system/sub-system being operated parallelly to the computer system 400. The processor 402 may include specialized processing units such as integrated system (bus) controllers, memory controllers/memory management control units, floating point units, graphics processing units, digital signal processing units, etc.

The processor 402 may be disposed in communication with one or more Input/Output (I/O) devices (411 and 412) via I/O interface 401. The I/O interface 401 may employ communication protocols/methods such as, without limitation, audio, analog, digital, stereo, IEEE®-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE® 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc. Using the I/O interface 401, the computer system 400 may communicate with one or more I/O devices 411 and 412.

In some embodiments, the processor 402 may be disposed in communication with a communication network 409 via a network interface 403. The network interface 403 may communicate with the communication network 409. The network interface 403 may employ connection protocols including, without limitation, direct connect, Ethernet (e.g., twisted pair 10/100/1000 Base T), Transmission Control Protocol/Internet Protocol (TCP/IP), token ring, IEEE® 802.11a/b/g/n/x, etc.

In an implementation, the communication network 409 may be implemented as one of the several types of networks, such as intranet or Local Area Network (LAN) and such within the organization. The communication network 409 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP) etc., to communicate with each other. Further, the communication network 409 may include a variety of network devices, including routers, bridges, servers, computing devices, storage devices, etc. In an embodiment, the communication network 409 may be used for interfacing with a database 105 for receiving data related to the one or more reference RNA strains 107 from earlier pandemic/epidemic.

In some embodiments, the processor 402 may be disposed in communication with a memory 405 (e.g., RAM 413, ROM 414, etc. as shown in FIG. 4) via a storage interface 404. The storage interface 404 may connect to memory 405 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 405 may store a collection of program or database components, including, without limitation, user/application interface 406, an operating system 407, a web browser 408, and the like. In some embodiments, computer system 400 may store user/application data 406, such as the data, variables, records, etc. as described in this invention. Such databases may be implemented as fault-tolerant, relational, scalable, secure databases such as Oracle® or Sybase®.

The operating system 407 may facilitate resource management and operation of the computer system 400. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM® OS/2®, MICROSOFT® WINDOWS® (XP®, VISTA/7/8, 10 etc.), APPLE® IOS®, GOOGLE™ ANDROID™, BLACKBERRY® OS, or the like.

The user interface 406 may facilitate display, execution, interaction, manipulation, or operation of program components through textual or graphical facilities. For example, the user interface 406 may provide computer interaction interface elements on a display system operatively connected to the computer system 400, such as cursors, icons, check boxes, menus, scrollers, windows, widgets, and the like. Further, Graphical User Interfaces (GUIs) may be employed, including, without limitation, APPLE® MACINTOSH® operating systems' Aqua®, IBM® OS/2®, MICROSOFT® WINDOWS® (e.g., Aero, Metro, etc.), web interface libraries (e.g., ActiveX®, JAVA®, JAVASCRIPT®, AJAX, HTML, ADOBE® FLASH®, etc.), or the like.

The web browser 408 may be a hypertext viewing application. Secure web browsing may be provided using Secure Hypertext Transport Protocol (HTTPS), Secure Sockets Layer (SSL), Transport Layer Security (TLS), and the like. The web browsers 408 may utilize facilities such as AJAX, DHTML, ADOBE® FLASH®, JAVASCRIPT®, JAVA®, Application Programming Interfaces (APIs), and the like. Further, the computer system 400 may implement a mail server stored program component. The mail server may utilize facilities such as ASP, ACTIVEX®, ANSI® C++/C #, MICROSOFT®, .NET, CGI SCRIPTS, JAVA®, JAVASCRIPT®, PERL®, PHP, PYTHON®, WEBOBJECTS®, etc. The mail server may utilize communication protocols such as Internet Message Access Protocol (IMAP), Messaging Application Programming Interface (MAPI), MICROSOFT® exchange, Post Office Protocol (POP), Simple Mail Transfer Protocol (SMTP), or the like. In some embodiments, the computer system 400 may implement a mail client stored program component. The mail client may be a mail viewing application, such as APPLE® MAIL, MICROSOFT® ENTOURAGE®, MICROSOFT® OUTLOOK®, MOZILLA® THUNDERBIRD®, and the like.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

Advantages of the embodiments of the present disclosure are illustrated herein.

In an embodiment, the present disclosure helps in an early identification of viral strains that have potential to turn into an epidemic. Consequently, the present disclosure helps to improve the response time in vaccine development and shortens time to market, thereby making the vaccines available for clinical trials earlier.

As stated above, it shall be noted that the method of the present disclosure may be used to overcome various technical problems related to predicting mutations in Ribonucleic acid (RNA) strains. In other words, the disclosed method has a practical application and provides a technically advanced solution to the technical problems associated with the existing mutations prediction system.

The aforesaid technical advancement and practical application of the proposed method may be attributed to the aspects of a) identifying one or more mutation sites for the new viral RNA strain by generating spatial nearness data corresponding to the one or more reference RNA strains based on comparison between the strain score of the new viral RNA strain and the one or more reference RNA strains and b) predicting one or more mutations of the new viral RNA strain by performing a generative modelling of a sequence of the new viral RNA strain with reference to the one or more mutation sites of the new viral RNA strain, as disclosed in steps 3 and 4 of the independent claims 1 and 10 of the present disclosure.

In light of the technical advancements provided by the disclosed method and the prediction system, the claimed steps, as discussed above, are not routine, conventional, or well-known aspects in the art, as the claimed steps provide the aforesaid solutions to the technical problems existing in the conventional technologies. Further, the claimed steps clearly bring an improvement in the functioning of the system itself, as the claimed steps provide a technical solution to a technical problem.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all the items are mutually exclusive, unless expressly specified otherwise. The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device/article is described herein (whether they cooperate), it will be clear that a single device/article may be used in place of the more than one device/article or a different number of devices/articles may be used instead of the shown number of devices or programs. The functionality and/or features of a device may be alternatively embodied by one or more other devices which are not explicitly described as having such functionality/features. Thus, other embodiments of invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference | |
|---|---|
| Number | Description |
| 101 | Prediction system |
| 103 | New viral RNA strain |
| 105 | Reference RNA strain |
| 107 | Database |
| 109 | Predicted mutations |
| 201 | I/O Interface |
| 203 | Processor |
| 205 | Memory |
| 207 | Data |
| 209 | Modules |
| 211 | Strain score |
| 213 | Spatial nearness data |
| 215 | Match score |
| 217 | Suffix tree |
| 219 | Infectivity score |
| 221 | Mortality score |
| 223 | Other data |
| 225 | Determining module |
| 227 | Calculating module |
| 229 | Identifying module |
| 231 | Predicting module |
| 233 | Other modules |
| 400 | Exemplary computer system |
| 401 | I/O Interface of the exemplary computer system |
| 402 | Processor of the exemplary computer system |
| 403 | Network interface |
| 404 | Storage interface |
| 405 | Memory of the exemplary computer system |
| 406 | User/Application |
| 407 | Operating system |
| 408 | Web browser |
| 409 | Communication network |
| 411 | Input devices |
| 412 | Output devices |
| 413 | RAM |
| 414 | ROM |

What is claimed is:

1. A method for predicting mutations in Ribonucleic acid (RNA) strains, the method comprising:
determining, by a prediction system, a similarity between a new viral RNA strain and one or more reference RNA strains;
calculating, by the prediction system, a strain score for the new viral RNA strain based on the similarity between the new viral RNA strain and the one or more reference RNA strains;
identifying, by the prediction system, one or more mutation sites for the new viral RNA strain by generating spatial nearness data corresponding to the one or more reference RNA strains based on comparison between the strain score of the new viral RNA strain and the one or more reference RNA strains; and
predicting, by the prediction system, one or more mutations of the new viral RNA strain by performing a generative modelling of a sequence of the new viral RNA strain with reference to the one or more mutation sites of the new viral RNA strain.

2. The method as claimed in claim 1, wherein determining the similarity between the new viral RNA strain and the one or more reference RNA strains comprises:
collecting, by the prediction system, the sequence of the new viral RNA strain and a sequence of the one or more reference RNA strains;
constructing, by the prediction system, a plurality of suffix trees corresponding to the sequence of the new viral RNA strain and sequence of each of the one or more reference RNA strains; and
determining, by the prediction system, a match score for the new viral RNA strain by comparing the plurality of suffix trees.

3. The method as claimed in claim 1, wherein calculating the strain score for the new viral RNA strain comprises:
collecting, by the prediction system, an infectivity metrics and a mortality metrics of the one or more reference RNA strains from earlier epidemics;
calculating, by the prediction system, an infectivity score by weighing a match score between a viral RNA sequence of the new viral RNA strain and a reference RNA sequence of the one or more reference RNA strains with the infectivity metrics;
calculating, by the prediction system, a mortality score by weighing the match score between the viral RNA sequence of the new viral RNA strain and the reference RNA sequence of the one or more reference RNA strains with the mortality metrics;
calculating, by the prediction system, a normalized infectivity score by dividing the infectivity score with a sum of the infectivity metric data for the one or more reference RNA strains;
calculating, by the prediction system, a normalized mortality score by dividing the mortality score with the sum of the mortality metric data for the one or more reference RNA strains;
determining, by the prediction system, the strain score for the one or more reference RNA strains by normalizing the normalized infectivity score and the normalized mortality score using a Euclidean norm, and wherein:
the infectivity metrics corresponds to a Basic Reproduction Number (R₀) data of the one or more reference RNA strains from the earlier epidemics; and
the mortality metrics corresponds to Case Fatality Ratio (CFR) data of strains from earlier epidemics, and wherein, calculating the strain score for the new viral RNA strain further comprises:
determining, by the prediction system, the one or more reference RNA strains that resemble the new viral RNA strain based on the match score and the strain score; and
identifying, by the prediction system, top contributing structural proteins in the new viral RNA strain responsible for current characteristics of the new viral RNA strain based on one or more match proportions, the match score and the strain score.

4. The method as claimed in claim 1, wherein identifying the one or more mutation sites comprises:
ordering, by the prediction system, sequences of each of the one or more reference RNA strains based on the spatial nearness data, wherein the spatial nearness data comprises a temporal similarity and a spatial similarity;
identifying, by the prediction system, a pattern of the temporal similarity and the spatial similarity using an Artificial Intelligence (AI) based attention transformer model; and
identifying, by the prediction system, the one or more mutation sites by identifying differences in the pattern.

5. The method as claimed in claim 1, wherein predicting the one or more mutations of the new viral RNA strain comprises:
collecting, by the prediction system, a plurality of human RNA Binding Protein (RBP) data that provides an experimental data on human RNA motifs that interact with one or more proteins;
predicting, by the prediction system, a mutated RNA sequence of the new viral RNA strain using a generative model, wherein the generative model takes the viral RNA sequence of the new viral RNA strain, information of possible mutation sites and the experimental data to generate a next generation of the new viral RNA strain with mutations; and
calculating, by the prediction system, a vibrational entropy, wherein an overall structural stability of the mutated RNA sequence is checked to identify one or more stable mutations.

6. A prediction system for predicting mutations in Ribonucleic acid (RNA) strains, the prediction system comprising:
a processor; and
a memory, communicatively coupled to the processor, wherein the memory stores processor-executable instructions, which, on execution, causes the processor (107) to:
determine a similarity between a new viral RNA strain and one or more reference RNA strains;
calculate a strain score for the new viral RNA strain based on the similarity between the new viral RNA strain and the one or more reference RNA strains;
identify one or more mutation sites for the new viral RNA strain by generating spatial nearness data corresponding to the one or more reference RNA strains based on comparison between the strain score of the new viral RNA strain and the one or more reference RNA strains; and
predict one or more mutations of the new viral RNA strain by performing a generative modelling of a sequence of the new viral RNA strain with reference to the one or more mutation sites of the new viral RNA strain.

7. The prediction system as claimed in claim 6, wherein determining the similarity between the new viral RNA strain and the one or more reference RNA strains comprises:
collecting the sequence of the new viral RNA strain and a sequence of the one or more reference RNA strains;
constructing a plurality of suffix trees corresponding to the sequence of the new viral RNA strain and sequence of each of the one or more reference RNA strains; and
determining a match score for the new viral RNA strain by comparing the plurality of suffix trees.

8. The prediction system as claimed in claim 6, wherein calculating the strain score for the new viral RNA strain comprises:
collecting an infectivity metrics and a mortality metrics of the one or more reference RNA strains from earlier epidemics;
calculating an infectivity score by weighing a match score between a viral RNA sequence of the new viral RNA strain and a reference RNA sequence of the one or more reference RNA strains with the infectivity metrics;
calculating a mortality score by weighing the match score between the viral RNA sequence of the new viral RNA strain and the reference RNA sequence of the one or more reference RNA strains with the mortality metrics;
calculating a normalized infectivity score by dividing the infectivity score with a sum of the infectivity metric data for the one or more reference RNA strains;
calculating a normalized mortality score by dividing the mortality score with the sum of the mortality metric data for the one or more reference RNA strains;
determining the strain score for the one or more reference RNA strains by normalizing the normalized infectivity score and the normalized mortality score using a Euclidean norm, and wherein:
the infectivity metrics corresponds to a Basic Reproduction Number (R₀) data of the one or more reference RNA strains from the earlier epidemics; and
the mortality metrics corresponds to Case Fatality Ratio (CFR) data of strains from earlier epidemics, and wherein, calculating the strain score for the new viral RNA strain further comprises:
determining the one or more reference RNA strains that resemble the new viral RNA strain based on the match score and the strain score; and
identifying top contributing structural proteins in the new viral RNA strain responsible for current characteristics of the new viral RNA strain based on one or more match proportions, the match score and the strain score.

9. The prediction system as claimed in claim 6, wherein identifying the one or more mutation sites comprises:
ordering sequences of each of the one or more reference RNA strains based on the spatial nearness data, wherein the spatial nearness data comprises a temporal similarity and a spatial similarity;
identifying a pattern of the temporal similarity and the spatial similarity using an Artificial Intelligence (AI) based attention transformer model; and
identifying the one or more mutation sites by identifying differences in the pattern.

10. The prediction system as claimed in claim 6, wherein predicting the one or more mutations of the new viral RNA strain comprises:
collecting a plurality of human RNA Binding Protein (RBP) data that provides an experimental data on human RNA motifs that interact with one or more proteins;
predicting a mutated RNA sequence of the new viral RNA strain using a generative model, wherein the generative model takes the viral RNA sequence of the new viral RNA strain, information of possible mutation sites and the experimental data to generate a next generation of the new viral RNA strain with mutations; and
calculating a vibrational entropy, wherein an overall structural stability of the mutated RNA sequence is checked to identify one or more stable mutations.

11. A non-transitory computer readable medium including instructions stored thereon that when processed by at least one processor, cause a prediction system to perform operations comprising:
determining a similarity between a new viral RNA strain and one or more reference RNA strains;
calculating a strain score for the new viral RNA strain based on the similarity between the new viral RNA strain and the one or more reference RNA strains;
identifying one or more mutation sites for the new viral RNA strain by generating spatial nearness data corresponding to the one or more reference RNA strains based on comparison between the strain score of the new viral RNA strain and the one or more reference RNA strains; and
predicting one or more mutations of the new viral RNA strain by performing a generative modelling of a sequence of the new viral RNA strain with reference to the one or more mutation sites of the new viral RNA strain.
